(19) United States
(12) Patent Application Publication
(10) Pub. No.: US 2007/0041915 A1
(43) Pub. Date: Feb. 22, 2007

(54) Use of a biguanide derivative for protecting skin against uvb radiation

(75) Inventors: Pierre Jean-Paul Potier, Paris (FR); Nobumichi-Andre Sasaki, Les Ulis (FR); Maria Conception Achab, Gif Sur Yvette (FR); Gisele Franck, Cachan (FR); Joanna Bakala, Paris (FR); Francoise Picot, Chevreuse (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: PHARMAMENS, Paris (FR)

(21) Appl. No.: 10/566,490

(22) PCT Filed: Jul. 29, 2004
(86) PCT No.: PCT/FR04/02041

§ 371 Date: Jan. 30, 2006

(30) Foreign Application Priority Data
Jul. 29, 2003 (FR) 0309306

(51) Int. Cl. A61K 8/49; A61K 31/445; A61K 31/40; A61K 31/155

(52) U.S. Cl. 424/59; 514/635; 514/317; 514/408

(57) ABSTRACT

The invention relates to the use of a bigaunide derivative for protecting skin against UVB radiation harmful effects and/or for protecting skin against undesirable and/or inaesthetic effects of UVB radiation.

The present invention relates to the use of a biguanide derivative for protecting the skin against the harmful effects of UVB radiation and/or for protecting the skin against: the adverse and/or displeasing effects of UVB radiation.

It is known that ultraviolet radiation (UV) having wavelengths of 280 nm to 400 nm derived from the sun and reaching the skin is of two types, namely UVA and UVB. Radiation with a wavelength of between 280nm and 320 nm, called UVB, is highly energetic but does not enter the skin to. great depth. It causes erythema and skin burns thereby preventing tan development. Its erythema power is 1000 times greater than that of UVA and its contrition towards the genesis of cancers is non-negligible.

It is also known that the sensitivity to sun rays varies greatly from one person to another. It is dependent, upon what is called the person's “phototype”. A difference must also be made between UV effects at usual doses in relation to the frequency of exposure. Exposure to UVA of average energy only leads to skin colouring, whereas exposure to UVB of average energy only leads to sunburn. On the other hand, long chronic exposures to UVB give rise to skin ageing and skin cancers. Over the long term the sun's rays are responsible for skin ageing (wrinkles, rosacea, skin thinning) and above all for skin cancers. 95% of these cancers are located at points the most often exposed to the sun. Severe sunburns in youth can lead to serious cancers in adult age.

Numerous sun filters are currently known. However, due to the increasingly greater need for such filters to afford sun protection while tanning, research into new products protecting the skin against UVB is always a current issue.

Surprisingly, the inventors have discovered that a derivative of biguanide, advantageously metformin, has a skin protecting effect against UVB.

Pharmaceutical compositions containing biguanides are already-known. They are used orally to treat certain forms of diabetes and chiefly Type II noninsulin-dependent diabetes as anti-hyperglycaemic agents promoting a return to glycaemic balance.

Metformin is the biguanide derivative that is most used for this type of treatment.

This medicinal product is administered by oral route in the form of tablets containing 500 mg, 850 mg or 1 g of active ingredient.

The daily dosage is between 1 and 2 g and is sometimes more.

Phase I clinical evaluation of metformin showed the absence of toxicity of the molecule examined at hypoglycaemic doses. Tolerance to the product proved to be good and its chronic toxicity practically zero. There is, no change in the behaviour or growth of animals; blood counts, uraemia and liver functions are not deteriorated.

The anti-hyperglycaemic effect of metformin is attributed firstly to the increased activity of endogenous insulin and secondly to the action of metformin via insulin-independent mechanisms. The action of metformin translates as reduced intestinal absorption of glucose, increased cell absorption of blood glucose and a reduction in the liver production of glucose (elimination of neoglucogenesis) and in the quantity of insulin required to normalise glycaemia. These effects result partly from the capability of metformin to amplify the action of existing insulin through an increase in the activity of the tyrosine kinase enzyme of the insulin receptor, which triggers the post-receptive, signalling response.

Metformin is also known in topical compositions to promote healing and is known to have angiogenesis action (FR 2 809 310).

In addition, some biguanide derivatives are also known as having an anti-inflammatory action (U.S. Pat. No. 4,163,800).

However, none of these documents neither describes nor suggests the use of a biguanide derivative to protect the skin against UVB rays.

The present invention therefore concerns the use of a biguanide derivative having the following-general formula I: 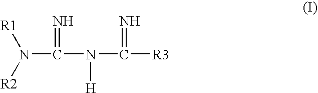
in which:

the R1 and R2 groups, independently of each other, represent a hydrogen atom, a C₁-C₇ alkyl group, a cycloalkyl group, a heterocycle, a C₂-C₇ alkenyl group, an aryl group, an aralkyl group, an aryloxylalkyl group or a heteroaryl group,

or R1 and R2 taken together represent a C₂-C₇ alkylene possibly containing one or more heteroatoms,

and the R3 group represents a primary, secondary or tertiary amine
or its pharmaceutically acceptable salt with the exception of the compound of formula: 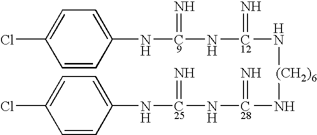
to manufacture a medicinal product intended. to protect the skin against the harmful effects of UVB radiation.

By the term “C₁-C₇ alkyl group” in the meaning of the present invention is meant any linear or branched C₁-C7 group for example the methyl, ethyl, propyl, isopropyl or butyl groups and their isomers.

By the term “cycloalkyl group” in the meaning of the present invention is meant any cycloalkyl group containing 3 to 7 carbon atoms, such as the cyclohexanyl group.

By the term “heterocycle” in the meaning of the present invention is meant any cycle containing 3 to 7 atoms, one or more of these being a heteroatom such as the atom of nitrogen, oxygen or sulphur, the others being carbon atoms.

By the term “C₂-C₇ alkenyl group” in the meaning of the present invention is meant any linear or branched C₂-C₇ alkenyl group such as the vinyl or allyl groups.

By the term “aryl group” in the meaning of the present invention is meant any hydrocarbonated aromatic group such as the phenyl group for example which may, contain one or more substituents such as for example a C₁-C₇ alkyl group such as defined above, a C₂-C₇ alkenyl group such as defined above or a halogen.

By the term “heteroaryl group” in the meaning of the present invention is meant any hydrocarbonated aromatic group. containing one or more heteroatoms such as atoms of nitrogen, oxygen or sulphur and able to carry one or more substituents such as for example a C₁-C₇ alkyl group such as defined above, a C₂-C₇ alkenyl group such as defined above, or a halogen. Examples of heteroaryl groups are the furyl, isoxazyl, pyridyl, pyrimidyl groups.

By the term “C₂-C₇ alkylene group” in the meaning of the present invention is meant any C₂-C₇ alkylene group such as the ethylene, trimethylene, tetramethylene or pentamethylene groups for example.

By the term “pharmaceutically acceptable salt” in the meaning of the present invention is meant any salt prepared from any non-toxic pharmaceutically acceptable acid, including organic and inorganic acids. Said acids include acetic, benzenesulphonic, benzoic, citric, ethanesulphonic, fumaric, gluconic, glutamic, hydrobromic, hydrochloric, lactic, maleic, malic, mandelic, methanesulphonic, mucic, nitric, pamoic, pantothenic, phosphoric, succinic, tartaric and paratoluenesulphonic acid. Advantageously hydrochloric acid is used.

In one embodiment of the invention, the medicinal product is intended to protect the skin against sunburn and skin cancers.

Advantageously the medicinal product has a protective action against the photo-immunosuppressive effect induced by UVB radiation on Langerhans cells.

In one particular embodiment of the invention the R3 group represents the secondary amine having the following formula:

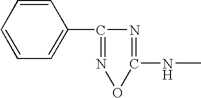

In one advantageous embodiment of the invention the R3 group represents NH₂.

In another embodiment of the invention the R1 and R2 groups, independently of each other, represent a hydrogen atom or a C₁-C₇ alkyl group.

Advantageously, the biguanide derivative is metformin, further advantageously in the form of a hydrochloride.

In particular the medicinal product may be in a pharmaceutical form for local use, advantageously of oil, cream, foam, liniment, lotion, ointment, liquid, gel, milk or spray type. The forms may , have a single-phase vehicle consisting of a neutral hydroxypropylcellulose gel or a gel containing sodium carboxymethylcellulose. It is also possible to prepare creams with two-phase vehicles comprising a hydrophilic phase dispersed in a lipophilic phase.

Advantageously the medicinal product contains 0.0.2 to 2% by weight of biguanide derivative having the general formula I or its pharmaceutically acceptable salt and a suitable excipient. These excipients may be chosen from among compounds having good compatibility with this active ingredient. They are for example hydrosoluble polymers of natural polymer type such as polysaccharides (xanthane gum, carouba gum, peptin, . . . ) or polypeptides, cellulose derivatives of methylcellulose, hydroxypropylcellulose, hydroxypropyl-methylcellulose type or further synthetic polymers, polaxamers, carbomers, PVA or PVP.

Finally, it is within the reach of all persons skilled in the art to add to this cosmetic composition various excipients of co-solvent type such as ethanol, glycerol, benzyl alcohol, humectants (glycerol), agents facilitating diffusion (transcurol, urea) or further anti-bacterial preserving agents (0.15% methyl p-hydroxybenzoate). It may also contain surfactants, stabilizers, emulsifiers, thickeners, other active ingredients imparting a complementary or possibly synergetic effect, trace elements, essential oils, perfumes, colourings, collagen, chemical or mineral filters, hydrating agents and spa waters.

Advantageously this sun protection medicinal product is in the form of an emulsion of oil-in-water type (i.e. a pharmaceutically acceptable carrier consisting of a continuous aqueous dispersing phase and a discontinuous oil dispersed phase) which, at various concentrations, contains the biguanide derivative of the present invention either alone or in association with one or more conventional organic filters whether lipophilic and/or hydrophilic, able to selectively. absorb harmful UV radiation, the biguanide derivative and optionally these filters (and their quantities) being chosen in relation to the desired sun protection factor (the sun protection factor being expressed mathematically as the ratio of the radiation time required to reach the erythematogenous threshold with the UV filter to the time required to reach the erythematogenous threshold with no UV filter). Also, mineral (nano) pigment(s) (by “nanopigments” is meant pigments whose mean primary particle size generally does not exceed 100 nm, this size preferably lying between 5 nm and 100 nm, and further preferably between 10 nm and 50 nm) containing metal oxides, titanium oxide in particular, may be used in the medicinal product of the present invention. It is known in particular that these substances, whether associated or not with usual UVA and/or UVB absorbing organic filters, are able to provide sun protection compositions containing the same with a certain extent of own or additional photoprotective properties, fairly limited however, by acting through -mere physical blocking of UV rays (reflection and/or radiation diffusing mechanisms).

For the purpose of improving the properties of the medicinal product of the present invention, it is also of interest to add thickening polymers to it having emulsifying properties, among which particular mention may be made of cross-linked copolymers of acrylic acid/acrylate alkyl in C₁₀-C₃₀ type such as those known under the trade mark “PEMULEN TR-1” and “CARBOPOL 1342” by Goodrich, which are currently the most frequently used.

In one particular embodiment of the invention, the biguanide derivative or its pharmaceutically acceptable salt is combined with at least one other active ingredient.

The present invention also concerns the cosmetic use of a biguanide derivative of the following general formula I: 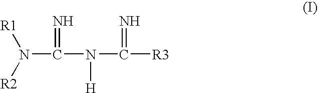
in which:

the R1 and R2 groups, independently of each other, represent a hydrogen atom, a C₁-C₇ alkyl group, a cylocalkyl group, a heterocycle, a C₂-C₇ alkenyl group, an aryl group, an aralkyl group, an aryloxylalkyl group or a heteroaryl group,

or R1 and R2 taken together represent a C₂-C₇ alkylene possibly containing one or more heteroatoms,

and the R3 group represents a primary, secondary or tertiary amine
or its pharmaceutically acceptable salt with the exception of the compound of formula: 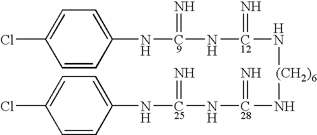
to protect the skin against the adverse and/or displeasing effects of UVB radiation such as sunburn for example or ageing (onset of wrinkles and age spots).

The examples given below of compositions of the invention and the examination of their activity are given by way of illustration and are not limitative.

### EXAMPLES

Several pharmaceutical forms were prepared with no preserving agent. The percentages are weight percentages.

### Example of Formulation 1

Metformin: 1%.
2.9% neutral hydroxypropylcellulose gel (Klucel by Aqualon type 99 MF EP): to final 100%.

### Example of Formulation 2

Metformin: 1%.
4.5% gel containing sodium carboxymethylcellulose (Aqualon): to final 100%.

### Example of Formulation 3

Metformin: 1% by weight with respect to the lipophilic phase.
33% (H/L) hydrocerin emulsion (fatty excipient by Roc® containing Vaseline, paraffin oil, triglycerides, ethers of polyoxyethylene and cerisine): to final 100%
   Study of the Protective Activity of an Ointment Containing Metformin Against an Immunosuppressive Effect on Langerhans Cells

The purpose of this study is to evidence the protective activity of metformin against an immunosuppressive photo effect (depletion of Langerhans cells) induced by UVB radiation on a survival-maintained human skin model.

The immunosuppressive photo activity induced by UVB is, assessed by counting the number of Langerhans cells in a separated epidermis and in tissue sections after anti-CDla labelling.

Operating Mode

Explants:

27 skin explants were prepared and maintained in-survival in a culture medium. They were divided among 9 batches of three explants: three reference batches, three excipient batches and three ointment batches containing 1% metformin (formulation example 3).

Irradiation:

3 batches (reference, excipient and ointment) were exposed to UVB radiation of 4 J/cm², three batches (reference, excipient and ointment) to UVB radiation of 6 J/cm² and the three last batches were placed in darkness during the time of irradiation.

Application of Products in Preventive Mode

Daily application of the ointment was 4 mg per explant for 3 days before irradiation.

Histology:

Analysis of the reference and treated explants was performed 24 h after irradiation.

Anti-CDla immunolabelling of Langerhans cells was made in the separated epidermis and in tissue sections.

Results

Non-Irradiated Reference Explants

The observed Langerhans cells are very large, highly dendritic rising high in the epidermis.

Reference Explants Irradiated With UVB at 4 J/cm2 and 6 J/cm2

The number of Langerhans cells is markedly lower with respect to the non-irradiated references. They have condensed cell bodies and show a strong-reduction in dendricity.

Explants Treated with the Excipients, Alone Irradiated at 4 J/cm² and 6 J/cm

The Langerhans cells show depletion. Their morphology is identical to the one visualized in the explants irradiated at 4 J/cm² and 6 J/cm² and non treated.

Explants Treated with the Ointment and Irradiated at 4 J/cm2 and 6 J/cm²

The number of Langerhans cells is higher in the explants irradiated and treated with the ointment containing metformin than in the non-treated irradiated explants. In addition, these cells have good dendritic form and their general morphology is close to that observed in the non-irradiated explants.

### CONCLUSION

Results of histological studies and in particular the immunolabelling of Langerhans cells in explants subjected to observations 24 hours after irradiation, show the protective action of metformin. When the ointment is applied preventively its protective activity is very significant and translates as a large number of Langerhans cells remaining intact.

On the basis of the results, the use of metformin can therefore be considered for the prevention of attack by sun radiation.

1. Method for protecting the skin of a patient against the harmful effects of UVB radiation comprising the administration to a patient in need thereof of an effective amount of a biguanide derivative of following general formula I: 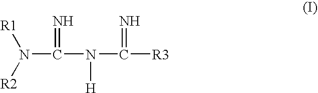
in which:
the R1 and R2 groups, independently of each other, represent a hydrogen atom, a C₁-C₇ alkyl group, a cycloalkyl group, a heterocycle, a C₂-C₇ alkenyl group, an aryl group, an aralkyl group, an aryloxylalkyl group or a heteroaryl group,
or R1 and R2 taken together represent a C₂-C₇ alkylene possibly containing one or more heteroatoms,
and the R3 group represents a primary, secondary or tertiary amine
or its pharmaceutically acceptable salt with the exception of the compound of formula:

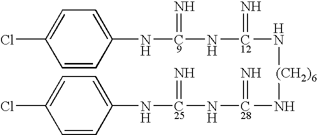

2. Method according to claim 1 wherein the harmful effects of UVB radiation are sunburn and skin cancers.

3. Method according to claim 1 wherein the harmful effects of UVB radiation are the photo immunosuppressive effect induced by UVB radiation on Langerhans cells.

4. Method according to claim 1, wherein the R1 and R2 groups, independently of each other, represent a hydrogen atom or a C₁-C₇ alkyl group.

5. Method according to claim 1 wherein the R3 group represents NH₂.

6. Method according to claim 5, wherein the derivative of biguanide is metformin.

7. (canceled)

8. (canceled)

9. Method according to claim 1, wherein the derivative of biguanide or its pharmaceutically acceptable salt is combined with at least one other active ingredient.

10. Method for protecting the skin against the adverse and/or displeasing effects of UVB radiation comprising the administration to a patient in need thereof of an effective amount of a biguanide derivative of following general formula I: 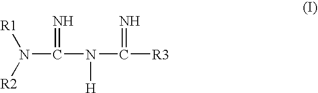
in which:
the R1 and R2 groups, independently of each other, represent a hydrogen atom, a C₁-C₇ alkyl group, a cycloalkyl group, a heterocycle, a C₂-C₇ alkenyl group, an aryl group, an aralkyl group, an aryloxylalkyl group or a heteroaryl group,
or R1 and R2 taken together represent a C₂-C₇ alkylene possibly containing one or more heteroatoms,
and the R3 group represents a primary, secondary or tertiary amine
or its pharmaceutically acceptable salt with the exception of the compound of formula:

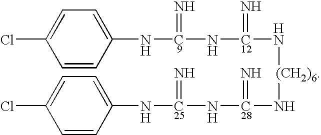

11. Method according to claim 6 wherein the metformin is in the form of a hydrochloride.

12. Method according to claim 1 wherein the biguanide derivative or its pharmaceutically acceptable salt are applied locally to the patient in need thereof.

13. Method according to claim 1 wherein the biguanide derivative or its pharmaceutically acceptable salt are used in the form of a medicinal product which contains a suitable excipient and wherein the amount of biguanide derivative or of its pharmaceutically acceptable salt in said medicinal product is of 0.02 to 2% by weight.
